(19) United States
(12) Patent Application Publication
(10) Pub. No.: US 2026/0294685 A1
(43) Pub. Date: Oct. 1, 2026

(54) LASER BEAM SCANNER SYSTEMS

(71) Applicant: ALCON INC., Fribourg (CH)

(72) Inventors: Lingfeng YU, Rancho Santa Margarita, CA (US); Steve X. CHEN, San Diego, CA (US); Mark Harrison FARLEY, Laguna Hills, CA (US)

(21) Appl. No.: 19/562,340

(22) Filed: Mar. 10, 2026

Related U.S. Application Data
(60) Provisional application No. 63/777,121, filed on Mar. 25, 2025.

(51) Int. Cl. A61F 9/008; A61B 17/00; A61B 18/20

(52) U.S. Cl. CPC A61F 9/008; A61B 2017/00057; A61B 2018/2025; A61B 2018/20359; A61F 2009/00897

(57) ABSTRACT

In one or more embodiments, a laser system includes a first beam scanner and a second beam scanner. The first beam scanner receives a first laser beam along a first source-to-scanner path and scans the first laser beam in a first coordinate direction. The first beam scanner receives a second laser beam along a second source-to-scanner path, where the second source-to-scanner path is at a source angle relative to the first source-to-scanner path, and scans the second laser beam in the first coordinate direction. The second beam scanner receives the first laser beam from the first beam scanner along a scanner-to-scanner path and scans the first laser beam in a second coordinate direction. The second beam scanner receives the second laser beam from the first beam scanner along the scanner-to-scanner path and scans the second laser beam in the second coordinate direction.

### FIELD

Embodiments of the present disclosure relate to laser beam scanner systems.

### BACKGROUND

Certain ophthalmic systems use different laser beams for different purposes. For example, a system may use one laser beam for surgical purposes and another laser beam for imaging purposes. The ophthalmic systems typically have a scanner system that can scan the laser beams for use during a procedure.

### SUMMARY

In one or more embodiments, a laser system includes a first laser source, a second laser source, a first beam scanner, and a second beam scanner. The first laser source provides a first laser beam, and the second laser source provides a second laser beam. The first beam scanner receives the first laser beam from the first laser source along a first source-to-scanner path and scans the first laser beam in a first coordinate direction. The first beam scanner receives the second laser beam from the second laser source along a second source-to-scanner path, where the second source-to-scanner path is at a source angle relative to the first source-to-scanner path, and scans the second laser beam in the first coordinate direction. The second beam scanner receives the first laser beam from the first beam scanner along a scanner-to-scanner path and scans the first laser beam in a second coordinate direction. The second beam scanner receives the second laser beam from the first beam scanner along the scanner-to-scanner path and scans the second laser beam in the second coordinate direction.

One or more embodiments may have none, one or more, any combination of, or all the following elements.

The source angle is in a range of 1 to 45 degrees.

The source angle is in a range of 1 to 20 degrees.

The first source-to-scanner path is at a first angle relative to the scanner-to-scanner path, where the first angle is in a range of 40 to 90 degrees, and the second source-to-scanner path is at a second angle relative to the scanner-to-scanner path, where the second angle is in a range of 90 to 150 degrees. The first angle may be in a range of 70 to 90 degrees. The second angle may be in a range of 90 to 120 degrees.

The first source-to-scanner path is substantially parallel to the scanner-to-scanner path, and the second source-to-scanner path is at an angle relative to the scanner-to-scanner path, where the angle is in a range of 40 to 90 degrees. The angle may be in a range of 70 to 90 degrees.

The first source-to-scanner path is substantially parallel to the scanner-to-scanner path, and the second source-to-scanner path is at an angle relative to the scanner-to-scanner path, where the angle is in a range of 90 to 150 degrees. The angle may be in a range of 90 to 120 degrees.

The first beam scanner rotates about a first axis, where a first plane is orthogonal to the first axis. The second beam path and the first beam path are located at the first plane.

At least one of the first laser beam or the second laser beam is a treatment laser beam.

At least one of the first laser beam or the second laser beam is an imaging laser beam.

The difference between a first wavelength center of the first laser beam and a second wavelength center of the second laser beam is less than 200 nanometers.

The difference between a first wavelength center of the first laser beam and a second wavelength center of the second laser beam is less than 100 nanometers.

The laser system further includes one or more optical devices that direct the first laser beam towards a target and direct the second laser beam towards the target.

In one or more embodiments, an ophthalmic system includes a first laser source, a second laser source, a flip mirror, a first beam scanner, a second beam scanner, and one or more optical devices. The first laser source provides a first laser beam, and the second laser source provides a second laser beam. At a first angular position, the flip mirror directs the first laser beam from the first laser source towards a first beam scanner. At a second angular position, the flip mirror directs the second laser beam from the second laser source towards the first beam scanner. The first beam scanner scans the first laser beam in a first direction and scans the second laser beam in the first direction. The second beam scanner receives the first laser beam from the first beam scanner and scans the first laser beam in a second direction. The second beam scanner receives the second laser beam from the second beam scanner and scans the second laser beam in the second direction. The one or more optical devices direct the first laser beam towards an eye and direct the second laser beam towards the eye.

One or more embodiments may have none, one or more, any combination of, or all the following elements.

The flip mirror comprises a dichroic mirror.

At least one of the first laser beam or the second laser beam is a treatment laser beam.

At least one of the first laser beam or the second laser beam is an imaging laser beam.

The difference between a first wavelength center of the first laser beam and a second wavelength center of the second laser beam is less than 200 nanometers.

### BRIEF DESCRIPTION OF THE DRAWINGS

FIG. 1 illustrates an example of an ophthalmic system with a laser system that directs a laser beam towards an eye, according to at least one embodiment described in the present disclosure;

FIG. 2 illustrates an example of a computing system, according to at least one embodiment described in the present disclosure;

FIGS. 3A, 3B, and 3C illustrate other examples of ophthalmic systems with laser systems that direct laser beams towards an eye, according to at least one embodiment described in the present disclosure;

FIG. 4 illustrates an example of a sensor that detects a laser source providing a laser beam, according to at least one embodiment described in the present disclosure; and

FIG. 5 illustrates another example of an ophthalmic system with a laser system that directs a laser beam towards an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

An ophthalmic system may use a first laser beam for surgical purposes and a second laser beam for imaging purposes. Known techniques for scanning the first and the second laser beams may be unsatisfactory. For example, a dichroic beam splitter may be used to separate the laser beams by wavelength to scan the laser beams separately. However, in some cases the center wavelengths of the first and the second laser beams may be too close to be scanned by a dichroic beam splitter.

One or more embodiments of the present disclosure may provide improvements over previous iterations of laser beam scanning systems. For example, a beam scanner can move through a first range of angles to scan a first laser beam and can move through a second range of angles to scan a second laser beam in order to scan multiple laser beams of any suitable wavelengths. As another example, the beam scanner may move through the first range of angles and the second range of angles to quasi-simultaneously scan the laser beams.

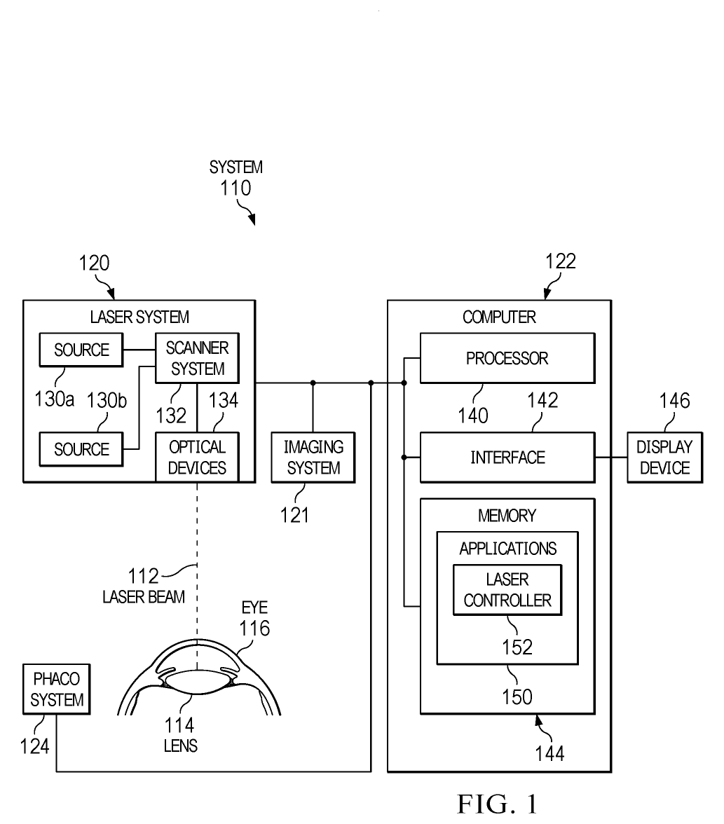

FIG. 1 illustrates an example of an ophthalmic system 110 with a laser system 120 that directs a laser beam 112 towards an eye 116, according to at least one embodiment described in the present disclosure. In the example, the ophthalmic system 110 includes the laser system 120, an imaging system 121, a computer system 122, and/or a phacoemulsification (phaco) system 124, which may be coupled (e.g., mechanically, electrically, and/or optically) as shown. The laser system 120 includes laser sources 130 (130a and 130b), a scanner system 132, and one or more optical devices 134. The computer system 122 includes a processor 140, an interface 142 (which may include a display device 146), and/or a memory 144, which may be coupled as shown. The memory 144 stores applications 150, such as a laser controller application 152.

According to an example of operation, the laser source 130a provides a first laser beam, and the laser source 130b provides a second laser beam. The scanner system 132 includes a first beam scanner and a second beam scanner. The first beam scanner receives the first laser beam from the first laser source along a first source-to-scanner path and scans the first laser beam in a first coordinate direction. The first beam scanner receives the second laser beam from the second laser source along a second source-to-scanner path, where the second source-to-scanner path is at an angle relative to the first source-to-scanner path, and scans the second laser beam in the first coordinate direction. The second beam scanner receives the first laser beam from the first beam scanner along a scanner-to-scanner path and scans the first laser beam in a second coordinate direction. The second beam scanner receives the second laser beam from the first beam scanner along the scanner-to-scanner path and scans the second laser beam in the second coordinate direction. The one or more optical devices 134 receive the first laser beam and the second laser beam and direct the laser beams towards the eye 116.

For ease of explanation, the embodiments may be described using the following example xyz-coordinate system, which may be regarded as the coordinate system of the ophthalmic system 110, although any suitable coordinate system may be used. In the example, the z-axis is aligned with the optical axis of the laser system 120, and an xy-plane is orthogonal to the z-axis.

The ophthalmic system 110 may be any suitable medical device that performs a medical procedure, such as a surgical and/or diagnostic procedure. In one or more embodiments, the ophthalmic system 110 may be a surgical system that directs a laser beam to a part of the eye (e.g., cornea, lens, and/or other eye tissue) to process the tissue (e.g., ablate and/or photodisrupt the tissue). In one or more embodiments, the ophthalmic system 110 may be a surgical system that performs cataract surgery (e.g., femtosecond laser assisted cataract surgery (FLACS)) to remove a crystalline lens from the eye. In FLACS, a laser (e.g. a femtosecond laser) may be used to create photodisruptions arranged in a fragmentation pattern to segment the crystalline lens in order to facilitate removal of the crystalline lens.

Turning to the components, the laser system 120 directs a laser beam 112 towards the eye 116. A laser source 130 (130a or 130b) generates the laser beam 112, and the scanner system 132 guides the laser beam 112, e.g., in the xy-plane. One or more optical devices 134 direct the laser beam 112 towards the eye 116. The laser beam 112 may have any suitable pulse duration, such as in the order of nanoseconds, picoseconds, femtoseconds, or attoseconds. The laser beam 112 may have any suitable wavelength, such as in the range of 150 nanometers (nm) to 20 micrometers (µm). Examples of ranges include the ultraviolet (e.g., in the range of 180 to 400 nm, such as 190 to 195 nm and/or 345 to 355 nm), visible, or infrared wavelength (e.g., in the range of 1050 to 1250 and/or 1250 to 1500 nm).

In one or more embodiments, the laser source 130a may provide a treatment laser beam designed to treat the eye 116, e.g., ablate, photo-disrupt, heat, or otherwise treat the eye 116. In an example, the treatment laser beam may be an ultraviolent or infrared femtosecond laser beam. In the one or more embodiments, the laser source 130b may provide an imaging laser beam designed to facilitate imaging of the eye 116. An imaging laser beam may be reflected from tissues of the eye 116 and allow an imaging system (e.g., an OCT system) to generate an image from the reflected light. In an example, the imaging laser beam may be an OCT beam (e.g., a short-pulsed laser beam) that allows an OCT imaging system to generate an OCT image of the eye 116.

In one or more embodiments, the difference between the wavelength center of the treatment laser beam and the wavelength center of the imaging laser beam may be less than 200 nanometers (nm), such as a difference in a range of 0 to 25 nm, 25 to 50 nm, 50 to 100 nm, and/or 100 to 200 nm. For example, the wavelength center of the treatment laser beam may be at 1030 nm, and the wavelength center of the imaging laser beam may be at 1055 nm, yielding a difference of 25 nm. In such an embodiment, the difference may be too small for a conventional dichroic beam splitter to be able to distinguish the laser beams. One or more embodiments of a scanner system 132 may allow for beam scanning that conventional dichroic beam splitters cannot perform, as described herein.

The scanner system 132 transversely and/or longitudinally directs the laser beam. The transverse direction refers to directions orthogonal to the direction of beam propagation, i.e., the x- and y-directions. The scanner system 132 may transversely direct the laser beam in any suitable manner, e.g., using a pair of galvanometrically-actuated scanner mirrors or an electro-optical crystal. The longitudinal direction refers to the direction of the laser beam propagation, e.g., the z-direction. The scanner system 132 may longitudinally direct the laser beam in any suitable manner, e.g., using a longitudinally adjustable lens, a lens of variable refractive power, or a deformable mirror that can control the z-position of the focal point. Examples of scanner systems 132 are described in more detail herein.

One or more optical devices 134 direct the laser beam along a beam path and may be located prior to, within, and/or after the scanner system 132. An optical device 134 can act on (e.g., transmit, reflect, refract, diffract, collimate, condition, shape, focus, modulate, and/or otherwise act on) a laser beam. Examples of optical devices 134 include a lens, prism, mirror, diffractive optical element (DOE), holographic optical element (HOE), and spatial light modulator (SLM).

The imaging system 121 includes one or more detectors that detect light reflected from the eye 116 to generate an image of the eye 116. A detector of the imaging system 121 may be any suitable detector that detects reflected light. For example, a detector may be a digital camera that records the image as digital image data. As another example, a detector may be an OCT detector that can image interference patterns to generate an OCT image.

The phaco system 124 removes the lens from the eye 116. In one or more embodiments, ultrasonic energy, laser energy, and/or other suitable energy is used to fragment, emulsify, and/or otherwise break up the lens. The phaco system 124 may remove the remaining lens pieces via, e.g., suction.

The computer system 122 performs operations to direct a laser beam towards the eye 116, including sending instructions to other components of the ophthalmic system 110 (e.g., the laser system 120) to perform operations (e.g., direct a laser beam towards the eye 116). The computer system 122 may use applications 150 to perform the operations. For example, the laser controller application 152 may be used to instruct the laser system 120 to generate a laser beam, scan the laser beam, and then direct the laser beam toward the eye 116. In one or more embodiments, the laser controller application 152 may be used to display a graphic user interface (GUI) on the display device 146. The GUI may include laser controller selectors that a user may use to select various parameters for customization of the laser beam.

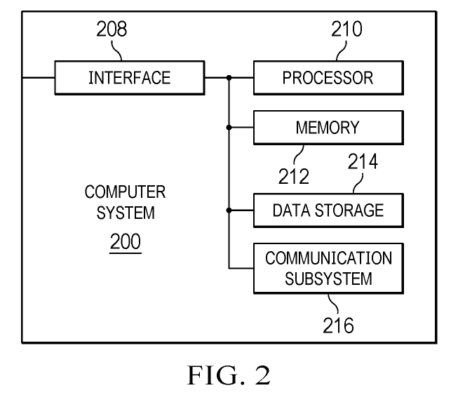

FIG. 2 illustrates an example of a computing system 200, according to at least one embodiment described in the present disclosure. The computing system 200 may include an interface 208, a processor 210, a memory 212, a data storage 214, and/or a communication subsystem 216, any or all of which may be communicatively coupled. Any or all of the computing system 200 may be implemented as computer hardware and/or software. One or more components of a computer system described herein may be implemented as described with reference to the computing system 200.

In the example, the interface 208 may receive input to the computing system 200 and/or send output from the computing system 200, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 200. Examples of user interfaces include displays, Graphical User Interfaces (GUIs), touchscreens, foot pedals, keyboards, joysticks, computer mouses (or mice), gesture sensors, microphones, and speakers.

Generally, the processor 210 may include any suitable special-purpose or general-purpose computer, computing entity, or processing device including various computer hardware or software modules and may be configured to execute instructions stored on any applicable computer-readable storage media. For example, the processor 210 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 2, the processor 210 may include any number of processors distributed across any number of network or physical locations that are configured to perform individually or collectively any number of operations described in the present disclosure.

The processor 210 may perform any suitable operations. In some embodiments, the processor 210 may interpret and/or execute program instructions and/or process data stored in the memory 212, the data storage 24, or the memory 212 and the data storage 214. In some embodiments, the processor 210 may fetch program instructions from the data storage 214 and load the program instructions into the memory 212. After the program instructions are loaded into the memory 212, the processor 210 may execute the program instructions, such as instructions to perform any of the methods disclosed herein, respectively.

The memory 212 and the data storage 214 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 210.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 210 to perform a certain operation or group of operations.

The communication subsystem 216 may include any component, device, system, or combination thereof that is configured to transmit, receive, and/or otherwise exchange information over a network in order to communicate with any suitable entity, such as with other devices at other locations or at the same location or even within the same system. The communication subsystem 216 may provide for communication among the devices described in the present disclosure, communication networks, computing devices, and other systems. For example, the communication subsystem 216 may allow the system 200 to communicate with other systems, such as other computing devices and/or networks. In some embodiments, the communication subsystem 216 may include a modem, a network card (wireless or wired), an optical communication device, an infrared communication device, a wireless communication device (such as an antenna), and/or chipset. Examples of communication subsystem 216 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the system 200 without departing from the scope of the present disclosure. For example, the system 200 may include more or fewer components than those explicitly illustrated and described.

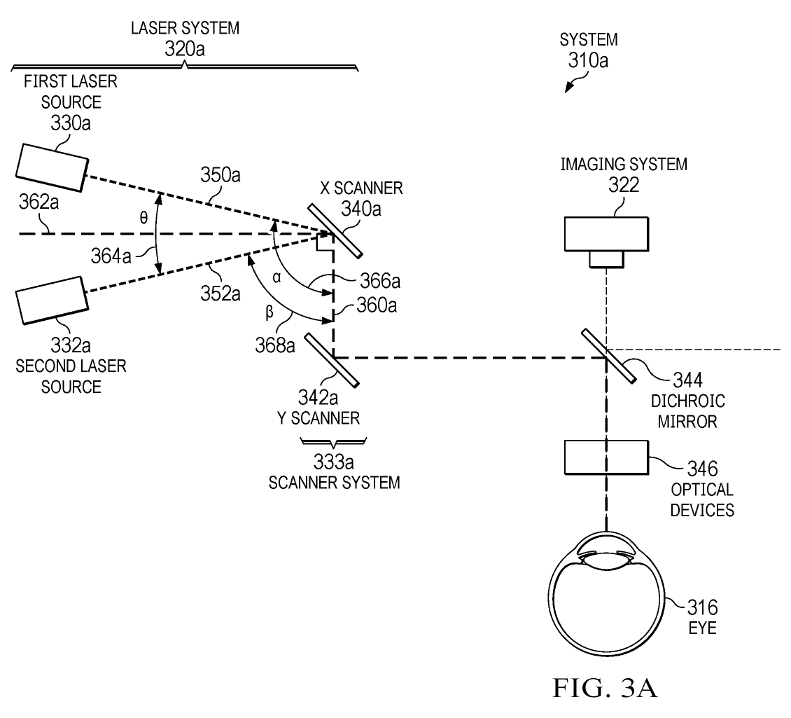

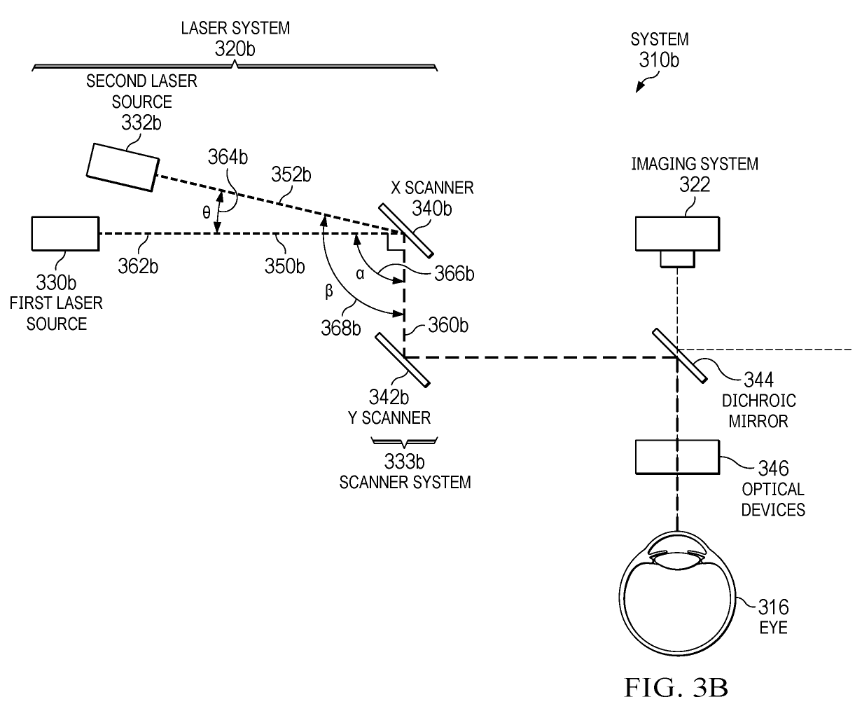

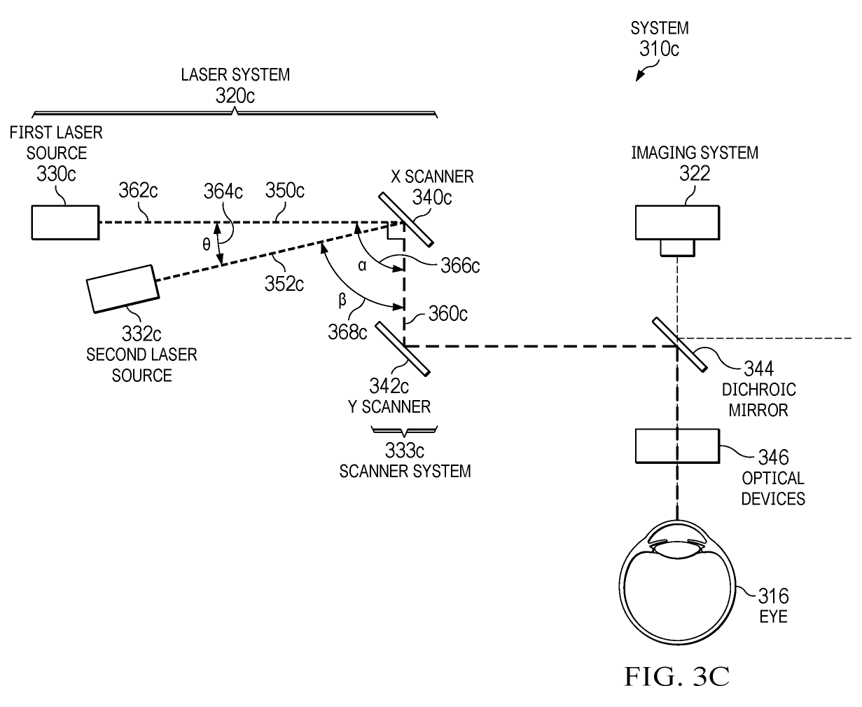

FIGS. 3A, 3B, and 3C illustrate examples of ophthalmic systems 310 (310a, 310b, and 310c, respectively) with laser systems 320 (320a, 320b, and 320c, respectively) that direct laser beams from first laser sources 330 (330a, 330b, and 330c, respectively) and/or second laser sources 332 (332a, 332b, and/or 332c, respectively) towards an eye 316, according to at least one embodiment described in the present disclosure. In the examples, an ophthalmic system 310 (310a, 310b, and/or 310c) may include a laser system 320 (320a, 320b, and/or 320c, respectively), an imaging system 322, a dichroic mirror 344, and/or optical devices 346, which may be coupled as shown. The laser system 320 (320a, 320b, and/or 320c) may include the first laser source 330 (330a, 330b, and/or 330c, respectively), the second laser source 332 (332a, 332b, and/or 332c, respectively), and/or a scanner system 333 (333a, 333b, and/or 333c), which may be coupled as shown. A scanner system 333 (333a, 333b, and/or 333c) may include an x-scanner 340 (340a, 340b, and/or 340c, respectively) and/or a y-scanner 342 (342a, 34b, and/or 342c, respectively), which may be coupled as shown.

In an example of operation, the first laser source 330 (330a, 330b, and/or 330c) provides a first laser beam (e.g., a treatment laser beam and/or an imaging laser beam), and the second laser source 332 (332a, 332b, and/or 332c, respectively) provides a second laser beam (e.g., an imaging laser beam and/or a treatment laser beam). When the x-scanner 340 (340a, 340b, and/or 340c) is moving through a first range of angles, the x-scanner 340 (340a, 340b, and/or 340c, respectively) scans the first laser beam in the x-direction and directs the first laser beam to the y-scanner 342 (342a, 342b, and/or 342c, respectively). When the x-scanner 340 (340a, 340b, and/or 340c) is moving through a second range of angles, the x-scanner 340 (340a, 340b, and/or 340c, respectively) scans the second laser beam in the x-direction and directs the second laser beam to the y-scanner 342 (342a, 342b, and/or 342c, respectively). Accordingly, the x-scanner 340 (340a, 340b, and/or 340c) can scan the first laser beam and the second laser beam (e.g., a treatment laser beam and an imaging laser beam) quasi-simultaneously.

Continuing the example, the y-scanner 342 (342a, 342b, and/or 342c) scans the first laser beam (or the second laser beam) in the y-direction. The dichroic mirror 344 directs the first laser beam (or the second laser beam) towards the optical devices 346, which direct the first laser beam (or the second laser beam) towards the eye 316. Light reflected from the eye 316 passes through the optical devices 346, and the dichroic mirror 344 passes the reflected light towards the imaging system 322, which generates an image of the eye 316 using the reflected light.

Turning to the components of the ophthalmic system 310 (310a, 310b, and/or 310c), the first laser source 330 (330a, 330b, and/or 330c, respectively) and the second laser source 332 (332a, 332b, and/or 332c, respectively) may be similar to the laser sources as described herein. In the examples, the first laser source 330 (330a, 330b, and/or 330c) directs the first laser beam along a first beam path 350 (350a, 350b, and/or 350c, respectively), e.g., a first source-to-scanner path. The second laser source 332 (332a, 332b, and/or 332c) directs the second laser beam along a second beam path 352 (352a, 352b, and/or 352c, respectively), e.g., a second source-to-scanner path. The first beam path 350 (350a, 350b, and/or 350c) and the second beam path 352 (352a, 352b, and/or 352c, respectively) may form a source angle, e.g., a theta angle 364 (364a, 364b, and/or 364c), that has the x-scanner 340 (340a, 340b, and/or 340c, respectively) at the vertex. This arrangement allows the x-scanner 340 (340a, 340b, and/or 340c, respectively) to scan the first laser beam when moving through the first range of angles and scan the second laser beam when moving through the second range of angles.

The x-scanner 340 (340a, 340b, and/or 340c) scans a laser beam in the x-direction, and the y-scanner 342 (342a, 342b, and/or 342c, respectively) scans the laser beam in the y-direction. The x-scanner 340 (340a, 340b, and/or 340c) and the y-scanner 342 (342a, 342b, and/or 342c) may be any suitable scanners. In the example, the x-scanner 340 (340a, 340b, and/or 340c) directs the laser beam toward the y-scanner 342 (342a, 342b, and/or 342c, respectively) along a scanner-to-scanner path 360 (360a, 360b, and/or 360c, respectively). A source-scanner axis 362 (362a, 362b, and/or 362c) may be defined as an axis orthogonal to the scanner-to-scanner path 360 (360a, 360b, and/or 360c, respectively) and in the plane of the first beam path 350 (350a, 350b, and/or 350c, respectively) and the second beam path 352 (352a, 352b, and/or 352c, respectively).

As described above, the x-scanner 340 (340a, 340b, and/or 340c) may move through a first range of angles to scan the first laser beam and then move through a second range of angles to scan the second laser beam to scan the first and the second laser beams quasi-simultaneously. The first laser beam and the second laser beam may be scanned in any suitable manner. In one or more embodiments, the first laser beam may be scanned for the same amount of time that the second laser beam is scanned, or the first laser beam may be scanned for a longer period of time than the second laser beam is scanned, or vice-versa. In one or more embodiments, a femtosecond treatment laser beam may be scanned for a longer period of time than an OCT imaging beam. For example, the femtosecond treatment laser beam may be scanned for 5 to 10 pulses, and then the OCT imaging beam may be scanned for 1 to 5 pulses.

In some embodiments, the ophthalmic systems 310 may be configured to intelligently time or interleave which of the first laser source 330 or the second laser source 332 is in operation. By using different durations and/or sequences of the first laser source 330 and/or the second laser source 332, the ophthalmic systems 310 may have the appearance of real-time visualization by interleaving occasional visualization scans with treatment scans, e.g., one visualization scan for every n treatment scans, where n may be in a range of 2 to 5, 5 to 10, 10 to 25, 25 to 50, and/or greater than 50. In some embodiments, there may be bursts of scans alternating between the visualization scans and the treatment scans, e.g., 2 to 10 treatment scans followed by 2 to 10 visualization scans, 10 to 100 treatment scans followed by 2 to 10 visualization scans, or any other number of scans. In some embodiments, the alternating scans may include more treatment scans followed by fewer visualization scans (e.g., 10 treatment scans followed by 2 visualization scans, or 100 treatment scans followed by 5 visualization scans). Additionally or alternatively, properties of the tissue being treated may be monitored using the visualization scans to provide feedback to a computing system during a laser treatment. For example, OCT scans interleaved with femtosecond laser pulses fragmenting or softening a cataractous lens may provide feedback of the extent of fragmentation or softening of the cataractous lens during the procedure. In some embodiments, the frequency with which the ophthalmic systems 310 switch back and forth between the first laser source 330 and/or the second laser source 332 may be based on a rate of change of the x-scanner 340 and/or the y-scanner 342.

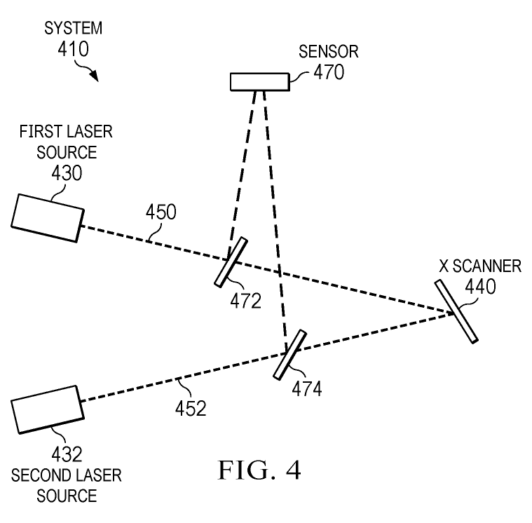

In one or more embodiments, a sensor may detect which laser source between the first laser source 330 (330a, 330b, and/or 330c) or the second laser source 332 (332a, 332b, and/or 332c, respectively) is providing a laser beam. In response, the x-scanner 340 (340a, 340b, and/or 340c, respectively) may be instructed to scan the laser beam from the first laser source 330 (330a, 330b, and/or 330c, respectively) or the second laser source 332 (332a, 332b, and/or 332c, respectively). The sensor may be any suitable sensor, such as an image sensor used in cameras, e.g., a complementary metal oxide semiconductor (CMOS) or a charge coupled device (CCD) image sensor. An example of such an embodiment is illustrated with reference to FIG. 4.

The dichroic mirror 344 directs the laser beams in one direction towards the eye 316 and directs light reflected from the eye 16 in another direction towards the imaging system 322. For example, the dichroic mirror 344 reflects the laser beams towards the eye 316 and transmits light reflected from the eye 16 towards the imaging system 322. The dichroic mirror 344 may be any suitable mirror that has different optical properties at different wavelengths. For instance, the dichroic mirror 344 may be reflective at one range of wavelengths and transmissive at a different range of wavelengths. The optical devices 346 may include any optical device that can direct a laser beam toward the eye 316, e.g., an objective lens. The imaging system 322 may be similar to imaging systems as described herein.

FIG. 3A illustrates an example of an arrangement of the first laser source 330a and the second laser source 332a, according to at least one embodiment described in the present disclosure. In the example, the first beam path 350a of the first laser source 330a and the second beam path 352a of the second laser source 332a form an angle theta 364a, with the x-scanner 340a at the vertex. The first beam path 350a forms an angle alpha 366a with the scanner-to-scanner path 360a, and the second beam path 352b forms an angle beta 368a with the scanner-to-scanner path 360a. The angle theta 364a may have any suitable value, such as a value between 1 and 45 degrees, e.g., 1 to 4 degrees, 4 to 8 degrees, 8 to 12 degrees, 12 to 20 degrees, 20 to 30 degrees, and/or 30 to 45 degrees. The angle alpha 366a may have any suitable value, such as a value between 90 and 150 degrees, e.g., 90 to 94 degrees, 94 to 98 degrees, 98 to 112 degrees, 112 to 120 degrees, and/or 120 to 150 degrees. The angle beta 368a may have any suitable value, such as a value between 40 and 90 degrees, e.g., 40 to 70 degrees, 70 to 78 degrees, 78 to 82 degrees, 82 to 86 degrees, and/or 86 to 90 degrees. While illustrated as having the first laser source 330a and the second laser source 332a symmetrically disposed about the source scanner axis 362a, it will be appreciated that they need not be.

FIG. 3B illustrates an example of an arrangement of the first laser source 330b and the second laser source 332b, according to at least one embodiment described in the present disclosure. In the example, the first beam path 350b forms an angle theta 364b with the second beam path 352b, with the x-scanner 340b at the vertex. The first beam path 350b forms an angle alpha 366b with the scanner-to-scanner path 360b, and the second beam path 352b forms an angle beta 368b with the scanner-to-scanner path 360b. The angle theta 364b may have any suitable value, as described herein. The angle alpha 366b may have any suitable value. For example, the first beam path 350b may be substantially parallel to the source-scanner axis 362b, e.g., the angle alpha 366b may have a value between 85 and 95 degrees (such as between 88 and 92 degrees). The angle beta 368b may have any suitable value, such as a value between 85 and 150 degrees, e.g., 85 to 90 degrees, 90 to 94 degrees, 94 to 98 degrees, 98 to 112 degrees, 112 to 120 degrees, and/or 120 to 150 degrees.

FIG. 3C illustrates an example of an arrangement of the first laser source 330c and the second laser source 332c, according to at least one embodiment described in the present disclosure. In the example, the first beam path 350c forms an angle theta 364c with the second beam path 352c, with the x-scanner 340c at the vertex. The first beam path 350c forms an angle alpha 366c with the scanner-to-scanner path 360c, and the second beam path 352c forms an angle beta 368c with the scanner-to-scanner path 360c. The angle theta 364c may have any suitable value, as described herein. The angle alpha 366c may have any suitable value. For example, the first beam path 350c may be substantially parallel to the source-scanner axis 362c, where the angle alpha 366c may have a value as described with reference to the angle alpha 366b. The angle beta 368c may have any suitable value, such as a value between 40 and 90 degrees, e.g., 40 to 70 degrees, 70 to 78 degrees, 78 to 82 degrees, 82 to 86 degrees, and/or 86 to 90 degrees.

FIG. 4 illustrates an example of a sensor 470 that detects whether a first laser source 430 or a second laser source 432 is providing a laser beam, according to at least one embodiment described in the present disclosure. The sensor 470 may then instruct the x-scanner 440 to scan the laser beam from the first laser source 430 or the second laser source 432, respectively, that is providing the laser beam. The sensor 470 may be any suitable sensor, such as an image sensor used in cameras, e.g., a complementary metal oxide semiconductor (CMOS) or a charge coupled device (CCD) image sensor.

In the example, an ophthalmic system 410 may include the first laser source 430, the second laser source 432, an x-scanner 440, the sensor 470, a beam splitter 472, and/or a beam splitter 474. The first laser source 430 directs a laser beam along a beam path 450 to the x-scanner 440. The beam splitter 472, which is located along the beam path 450, directs a portion of the laser beam towards the sensor 470 and allows the rest of laser beam to travel to the x-scanner. Similarly, the second laser source 432 directs a laser beam along a beam path 452 to the x-scanner 440. The beam splitter 474, which is located along the beam path 452, directs a portion of the laser beam towards the sensor 470 and allows the rest of the laser beam to travel to the x-scanner.

The sensor 470 receives a laser beam and may detect whether the first laser source 430 or the second laser source 432 is providing the laser beam in any suitable manner. In one or more embodiments, the laser beam interacts with the sensor 470 and yields a laser spot on the sensor, and the sensor 470 identifies the first laser source 430 or the second laser source 432 according to a feature of the laser spot. For example, the sensor 470 may identify the first laser source 430 or the second laser source 432 according to the size, intensity, position, wavelength, pulse duration, and/or other feature of the laser spot.

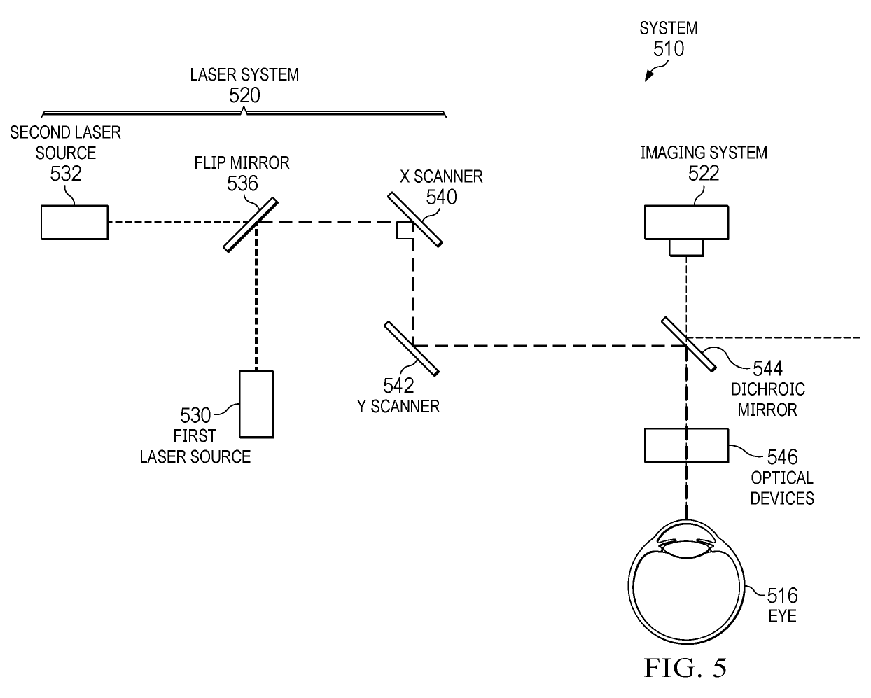

FIG. 5 illustrates an example of an ophthalmic system 510 with a laser system 520 that directs a laser beam from a first laser source 530 and/or a second laser source 532 towards an eye 516, according to at least one embodiment described in the present disclosure. In the example, the ophthalmic system 510 includes a laser system 520, an imaging system 522, a dichroic mirror 544, and/or optical devices 546, which may be coupled as shown. The laser system 520 may include a first laser source 530, a second laser source 532, a flip mirror 536, an x-scanner 540, and/or a y-scanner 542, which may be coupled as shown.

In an example of operation, the first laser source 530 provides a first laser beam (e.g., a treatment laser beam and/or an imaging laser beam), and the second laser source 532 provides a second laser beam (e.g., an imaging laser beam and/or a treatment laser beam). When in a first position, the flip mirror 536 transmits the first beam to the x-scanner 540, which scans the first beam in the x-direction. The y-scanner 542 scans the first laser beam in the y-direction, and the dichroic mirror 544 directs the first laser beam towards the optical devices 546, which direct first laser beam towards the eye 516. When in a second position, the flip mirror 536 transmits the second beam to the x-scanner 540, which scans the second beam in the x-direction. The y-scanner 542 scans the second laser beam in the y-direction, and the dichroic mirror 544 directs the second laser beam towards the optical devices 546, which direct second laser beam towards the eye 516. Light reflected from the eye 516 passes through the optical devices 546, and the dichroic mirror 544 transmits the reflected light towards the imaging system 522, which generates an image of the eye 516 using the reflected light.

Turning to the components of the ophthalmic system 510, the first laser source 530, the second laser source 532, the x-scanner 540, the y-scanner 542, the imaging system 522, the dichroic mirror 544, and/or the optical devices 546 may be as described herein. The flip mirror 536 may be any suitable flip mirror, such as a two-position, high-speed flip mirror.

In some embodiments, the ophthalmic system 510 may be configured to intelligently time or interleave which of the first laser source 530 or the second laser source 532 is in operation. By using different durations and/or sequences of the first laser source 530 and/or the second laser source 532, the ophthalmic system 510 may have the appearance of real-time visualization by interleaving occasional visualization scans with treatment scans, e.g., one visualization scan for every n treatment scans, where n may be in a range of 2 to 5, 5 to 10, 10 to 25, 25 to 50, and/or greater than 50. In some embodiments, there may be bursts of scans alternating between the visualization scans and the treatment scans, e.g., 2 to 10 treatment scans followed by 2 to 10 visualization scans, 10 to 100 treatment scans followed by 2 to 10 visualization scans, or any other number of scans. In some embodiments, the alternating scans may include more treatment scans followed by fewer visualization scans (e.g., 10 treatment scans followed by 2 visualization scans, or 100 treatment scans followed by 5 visualization scans). Additionally or alternatively, properties of the tissue being treated may be monitored using the visualization scans to provide feedback to a computing system during a laser treatment. For example, OCT scans interleaved with femtosecond laser pulses fragmenting or softening a cataractous lens may provide feedback of the extent of fragmentation or softening of the cataractous lens during the procedure. In some embodiments, the frequency with which the ophthalmic system 510 switches back and forth between the first laser source 530 and/or the second laser source 532 may be based on a rate of change of the flip mirror 536.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. A laser system comprising:
a first laser source configured to provide a first laser beam;
a second laser source configured to provide a second laser beam;
a first beam scanner configured to:
receive the first laser beam from the first laser source along a first source-to-scanner path;
scan the first laser beam in a first coordinate direction;
receive the second laser beam from the second laser source along a second source-to-scanner path, the second source-to-scanner path at a source angle relative to the first source-to-scanner path; and
scan the second laser beam in the first coordinate direction; and
a second beam scanner configured to:
receive the first laser beam from the first beam scanner along a scanner-to-scanner path;
scan the first laser beam in a second coordinate direction;
receive the second laser beam from the first beam scanner along the scanner-to-scanner path; and
scan the second laser beam in the second coordinate direction.

2. The laser system of claim 1, the source angle in a range of 1 to 45 degrees.

3. The laser system of claim 1, the source angle in a range of 1 to 20 degrees.

4. The laser system of claim 1:
the first source-to-scanner path at a first angle relative to the scanner-to-scanner path, the first angle in a range of 40 to 90 degrees; and
the second source-to-scanner path at a second angle relative to the scanner-to-scanner path, the second angle in a range of 90 to 150 degrees.

5. The laser system of claim 4, the first angle in a range of 70 to 90 degrees.

6. The laser system of claim 4, the second angle in a range of 90 to 120 degrees.

7. The laser system of claim 1:
the first source-to-scanner path substantially parallel to the scanner-to-scanner path; and
the second source-to-scanner path at an angle relative to the scanner-to-scanner path, the angle in a range of 40 to 90 degrees.

8. The laser system of claim 7, the angle in a range of 70 to 90 degrees.

9. The laser system of claim 1:
the first source-to-scanner path substantially parallel to the scanner-to-scanner path; and
the second source-to-scanner path at an angle relative to the scanner-to-scanner path, the angle in a range of 90 to 150 degrees.

10. The laser system of claim 9, the angle in a range of 90 to 120 degrees.

11. The laser system of claim 1:
the first beam scanner configured to rotate about a first axis, a first plane orthogonal to the first axis; and
the second beam path and the first beam path located at the first plane.

12. The laser system of claim 1, at least one of the first laser beam or the second laser beam being a treatment laser beam.

13. The laser system of claim 1, at least one of the first laser beam or the second laser beam being an imaging laser beam.

14. The laser system of claim 1, a difference between a first wavelength center of the first laser beam and a second wavelength center of the second laser beam being less than 200 nanometers.

15. The laser system of claim 1, a difference between a first wavelength center of the first laser beam and a second wavelength center of the second laser beam being less than 100 nanometers.

16. The laser system of claim 1, further comprising a sensor configured to:
receive a laser beam;
detect that the laser beam comprises the first laser beam; and
instruct the first beam scanner to scan the first laser beam in the first coordinate direction.

17. The laser system of claim 1, further comprising a sensor configured to:
receive a laser beam;
detect that the laser beam comprises the second laser beam; and
instruct the first beam scanner to scan the second laser beam in the first coordinate direction.

18. An ophthalmic system comprising:
a first laser source configured to provide a first laser beam;
a second laser source configured to provide a second laser beam;
a flip mirror configured to:
at a first angular position, direct the first laser beam from the first laser source towards a first beam scanner;
at a second angular position, direct the second laser beam from the second laser source towards the first beam scanner;
the first beam scanner configured to:
scan the first laser beam in a first direction; and
scan the second laser beam in the first direction; and
a second beam scanner configured to:
receive the first laser beam from the first beam scanner;
scan the first laser beam in a second direction;
receive the second laser beam from the second beam scanner; and
scan the second laser beam in the second direction; and
one or more optical devices configured to:
direct the first laser beam towards an eye; and
direct the second laser beam towards the eye.

19. The ophthalmic system of claim 18, the flip mirror comprising a dichroic mirror.

20. The ophthalmic system of claim 18, at least one of the first laser beam or the second laser beam being a treatment laser beam.

21. The ophthalmic system of claim 18, at least one of the first laser beam or the second laser beam being an imaging laser beam.

22. The ophthalmic system of claim 18, a difference between a first wavelength center of the first laser beam and a second wavelength center of the second laser beam being less than 200 nanometers.
